(19) United States
(12) Patent Application Publication
(10) Pub. No.: US 2016/0317816 A1
(43) Pub. Date: Nov. 3, 2016

(54) Optimization of Pacemaker Settings with Electrogram

(71) Applicant: AtCor Medical Pty Ltd, West Ryde (AU)

(72) Inventors: Dean Carl Winter, Portland, OR (US); Douglas Todd Kurschinski, Wheaton, IL (US)

(73) Assignee: AtCor Medical Pty Ltd, West Ryde (AU)

(21) Appl. No.: 15/141,923

(22) Filed: Apr. 29, 2016

Related U.S. Application Data
(60) Provisional application No. 62/154,153, filed on Apr. 29, 2015.

(51) Int. Cl. A61N 1/365; A61N 1/368; A61N 1/372; A61N 1/05

(52) U.S. Cl. CPC A61N 1/36585; A61N 1/056; A61N 1/36564; A61N 1/3684; A61N 1/37247

(57) ABSTRACT

The system provides information to facilitate efficient optimization of programmer settings for cardiac pacemakers. It simultaneously measures a patient's electrogram (EGM) and peripheral blood pressure (or volumetric displacement) waveform in order to calculate, in real-time and non-invasively, a value correlated to the pre-ejection time (PET) and, optionally, ejection duration (ED) for the patient's left ventricle. The peripheral pulse waveform can be monitored with a wrist mounted tonometer, or a suitable brachial cuff device. The time difference between the occurrence of the first detected positive or negative peak in a patient's LV electrogram trace (EGM) and the foot of the pulse on the peripheral pulse waveform is defined as a surrogate pre-ejection time interval (SPET). Data including the electrogram and peripheral pulse trace, as well as the calculated, surrogate pre-ejection time interval (SPET) for each heart beat and trending is displayed on a computer monitor, thereby allowing a physician or nurse to quickly optimize PET for the patient and adjust programmer settings for an implanted pacemaker.

### FIELD OF THE INVENTION

The invention relates to the optimization of programmable settings for cardiac pacemakers. It uses simultaneous measurement of a patient's left ventricular electrogram (LV electrogram or EGM) and peripheral blood pressure waveform in order to calculate, in real-time, a value correlated to the pre-ejection time (PET) for the patient's left ventricle. The LV electrogram represents local ventricular depolarization and is acquired directly or indirectly from a pacemaker lead implanted in the left ventricle. More specifically, the time between the detection of depolarization in the left ventricle and the detection of the foot of the pressure pulse in the peripheral pressure waveform is calculated and displayed, and available to be used by a physician or nurse to quickly optimize PET for the patient when adjusting programmable settings for an implanted pacemaker. The system is also able to determine ejection duration (ED) for the patient's left ventricle.

### BACKGROUND

A biventricular pacemaker is a type of cardiac pacemaker that can pace both the right and the left ventricle (typically the lateral wall of the left ventricle). By pacing both right and left ventricles, the pacemaker is able to resynchronize a heart whose opposing walls and right and left ventricles do not contract in synchrony. Biventricular pacemakers have at least two leads, one in the right ventricle to stimulate the septum, and the other inserted through the coronary sinus to pace the lateral wall of the left ventricle. There is typically also a lead in the right atrium to facilitate synchrony with atria contraction. The use of a biventricular pacemaker is generally referred to as cardiac resynchronization therapy (CRT). Some pacemakers have more than three or four pacing/sensing leads.

Programmable biventricular pacemakers enable optimization of the various time delays between pacemaker timing pulses. This optimization procedure requires the physician or nurse to set delays between the various timing pulses. Its general purpose is to coordinate contraction of the various chambers in the heart to improve overall efficiency and function. The onset of electrical cardiac activity in an electrocardiogram (ECG) is marked by the onset of the QRS complex and corresponds generally to the initial impulse time (T₀) for the contracting left ventricle. The time from the onset of the Q-wave to the closure of the mitral valve often is termed electromechanical delay (EMD). The isovolumetric contraction time interval (IVCT) begins when the mitral valve closes and ends when the blood pressure within the left ventricle is sufficient to open the aortic valve. The combination of EMD and IVCT is referred to in the art as the pre-ejection time interval (PET), and is a particularly useful parameter for CRT optimization. Typically, the attending physician will want to minimize PET.

Two methods of optimizing settings in programmable cardiac pacemakers are disclosed in U.S. Pat. No. 8,112,150, entitled “Optimization of Pacemaker Settings”, and U.S. Pat. No. 9,220,903 entitled “Optimization of Pacemaker Settings with R-Wave Detection”, incorporated herein by reference and assigned to the Assignee of the present application, AtCor Medical Pty. Ltd. The inventions in Assignee's '150 patent and '903 patent use simultaneous measurement of a patient's electrocardiogram (ECG) and a patient's peripheral blood pressure waveform in order to calculate, in real-time and non-invasively, a value correlated to the pre-ejection time (PET) for the patient's left ventricle. This value is termed a surrogate pre-ejection time (SPET) and its calculation and display enables a physician or nurse to quickly optimize PET by adjusting the programmable settings for the implanted pacemaker. To be more specific, in the systems disclosed in the '150 patent and '903 patent, the electrocardiogram is analyzed for each pulse to determine the exact time (T₀) corresponding to the onset of the QRS complex, or the time that the Q-wave reaches its minimum value or the R-wave reaches its maximum value as an approximation to the onset of the QRS complex. The system also measures the patient's peripheral pressure waveform using for example a tonometer at the wrist or a brachial volumetric waveform using a brachial cuff as disclosed in U.S. Pat. No. 9,314,170 entitled “Brachial Cuff” by Ahmad Qasem, assigned to the assignor of the present application and incorporated herein by reference. The opening of the aortic valve is marked by an abrupt rise of pressure in the aorta which results in a pressure pulse waveform rising to a peak systolic pressure and then declining. The arrival of the foot of the pressure waveform at the peripheral artery, e.g. a radial artery, is delayed by a transit time (K) for the pressure wave to travel from the aorta to the peripheral artery. For any individual patient, the travel distance for the pressure wave from the aorta to the peripheral location remains constant when the patient is at rest during the CRT optimization session, as long as the peripheral pressure is measured at a fixed location (e.g. at a fixed location on the user's wrist to the measure the pressure waveform at the radial artery or a brachial cuff to measure the volumetric waveform at the brachial artery). Testing indicates that the assumption that the pulse wave velocity for the patient remains constant over the time frame required for CRT optimization is quite accurate as long as the patient remains at rest. In the '150 patent, the time interval between the Q-wave (T₀) in the electrocardiogram and the foot (T₂) of the peripheral pressure wave, when the ECG trace and radial waveform are measured simultaneously, represents the actual pre-ejection time interval (PET) plus a fixed value (K), which are combined as described in the '150 patent to calculate a surrogate pre-ejection time (SPET). In the '903 patent, the time interval between the R-wave (T_{R}) in the electrocardiogram and the foot (T₂) of the peripheral pressure wave is used to calculate SPET. Since there is a constant offset (K) between the actual PET and the surrogate SPET, the doctor or nurse can optimize the pacemaker settings to minimize the actual pre-ejection time PET by minimizing the surrogate pre-ejection time SPET.

### SUMMARY OF THE INVENTION

One purpose of the invention is to avoid the need to measure a patient's electrocardiogram (ECG) while measuring the patient's peripheral pulse waveform, e.g., a radial pressure waveform with a tonometer or a brachial volumetric waveform with a brachial cuff, when calculating the surrogate pre-ejection time SPET. Instead, the invention uses the patient's LV electrogram to detect local ventricular depolarization when calculating the surrogate pre-ejection time SPET. The use of an electrocardiogram (ECG) requires the use of multiple sensors on the chest and torso of the patient to provide a generalized signal representing the electrical activity of the heart. In contrast, the invention uses the signal generated by a pacing/sensing lead of the cardiac pacemaker implanted in the patient's left ventricle muscle. The signal sensed by the lead produces an LV electrogram which is a plot of local electrical depolarization of the left ventricle as a function of time. A deflection in the time plot of the depolarization on the LV electrogram coincides with the onset of ventricular contraction. The invention thus uses an existing signal, and avoids the need for an electrocardiogram.

For each respective pulse, the LV electrogram is analyzed to determine a time correlating to the deflection, and this time is defined as an LV impulse time (T₀) for the contracting ventricle. The time (T₂) corresponding to the realization of systolic onset in the detected peripheral pulse waveform is also determined for each respective pulse. In the preferred embodiment of the invention, time T₂ corresponding to the onset of systole in the measured peripheral pulse waveform is determined by analyzing the first derivative of the peripheral pulse waveform and identifying a first negative to positive zero crossing preceding a maximum value for the first derivative. In accordance with the invention, the time values T₀ and T₂ are used to calculate a surrogate pre-ejection time interval (SPET) for the pulse. This information (SPET), and trends of this information, can be used conveniently by a medical staff in order to optimize CRT adjustments.

In another aspect, the invention pertains to a system which includes hardware components and software tools that are particularly well suited to conveniently assist medical staff during CRT optimization by providing information relating to the patient's surrogate pre-ejection time (SPET). The preferred system uses much of the same hardware that is currently available in a SphygmoCor® system, utilizing an MM3™ digital signal processing electronic module. Data from the pacemaker, namely the LV electrogram, are transmitted to the electronics module as is data from the pressure sensor for the peripheral pulse waveform, e.g. data from a tonometer or filtered data from a pressure sensor in a brachial cuff device. Analog data is sent from the electronics module to an A/D converter and the resulting digital data is analyzed and displayed, e.g., via a programmed personal computer or other programmable device. Traces of the LV electrogram data and the peripheral pulse waveform data are displayed as a function of time, and in real-time. The software allows the attending staff to select a given series of data representing a series of heart beats for which the surrogate pre-ejection time (SPET) is calculated for each pulse. The system preferably displays the data for each heart beat as well as an average and standard deviation for the selected series of heart beats. The system also allows the user to store data for later analysis. Typically, attending staff would adjust settings for the programmable pacemaker during CRT optimization, and compare SPET data from a previous setting to the current setting in an attempt to optimize (e.g. minimize) SPET. If desired, the system can also calculate and display other additional parameters as well. For example, as an optional feature, the system determines and displays ejection duration (ED) calculated from the peripheral pulse waveform, as is known in the art.

Further objects, features and advantages of the invention will be apparent from the following drawings and detailed description thereof.

### BRIEF DESCRIPTION OF THE DRAWINGS

FIG. 1 is a schematic diagram illustrating blood flow through a patient's left ventricle as well as a corresponding typical LV electrogram and radial artery pressure pulse waves.

FIG. 2 is a schematic drawing illustrating a system constructed in accordance with an embodiment of the invention.

FIG. 2A is a schematic drawing of a cardiac pacemaker having multiple pacing/sensing leads, with one lead implanted in the patient's left ventricle.

FIG. 3 is a representative screen display on a personal computer in a system implementing the invention.

FIG. 4 is a graphical screen display similar to that shown in FIG. 3, however, FIG. 3 is intended to represent an example of data displayed prior to CRT optimization, whereas FIG. 4 is intended to represent data displayed after CRT optimization.

FIG. 5 is a view illustrating the use of a wrist-mounted tonometer.

### DETAILED DESCRIPTION OF THE DRAWINGS

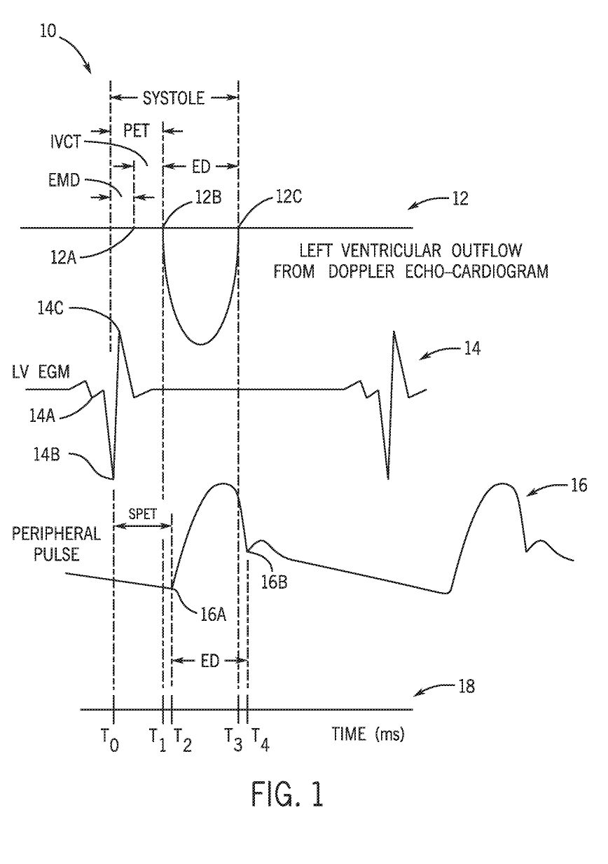

FIG. 1 is a schematic plot 10 with a variety of illustrative data relating to a hypothetical patient's cardiac cycle. In FIG. 1, curve 12 illustrates the outflow of blood from the patient's left ventricle as a function of time measured via a Doppler echo-cardiogram. Curve 14 represents an LV electrogram trace (EGM). The width of the deflections corresponding to local depolarization in the LV electrogram trace 14 is exaggerated to facilitate the explanation of the invention. Curve 16 in FIG. 1 is a peripheral pulse waveform, for example a waveform of the pressure pulse taken at the patient's radial artery using a tonometer. Alternatively, the peripheral pulse waveform can be volumetric displacement waveform taken with a brachial cuff device, where the pressure of the brachial cuff around the arm of the patient is held constant, and the analog signal from the pressure sensor in the cuff device has been filtered with suitable high pass and low pass filter to preserve the cardiovascular features of the volumetric pulse waveform as disclosed in the above incorporated U.S. Pat. No. 9,314,170 entitled “Brachial Cuff”. In accordance with the invention, the patient's LV electrogram 14 and peripheral pulse waveform 16 are measured simultaneously. The horizontal axis at the bottom of FIG. 1 represents time in milliseconds. A typical cycle for a heart beat might take one second or one thousand milliseconds.

A full cardiac cycle is divided into systole, which corresponds to contraction of the ventricles, and diastole which corresponds to the relaxation of the ventricles. In general terms, systole includes a pre-ejection time (PET) interval, and an ejection duration (ED), which is the amount of time that the aortic valve is open during the cycle. The pre-ejection time (PET) consists of 1) electromechanical delay (EMD) which is typically defined as the time interval from the onset of the Q-wave in an electrocardiogram (ECG) to the onset of physical cardiac contraction 12A; plus 2) the isovolumetric contraction time (IVCT), which is the initial period of ventricular contraction after the mitral valve closes but before the aortic valve opens. In accordance with an embodiment of the present invention, the system detects the first peak value 14B or 14C in the LV electrogram 14. This time is designated as T₀ on axis 18 in FIG. 1. As is illustrated by curve 12, there is no blood outflowing from the left ventricle during isovolumetric contraction (IVCT). As the pressure within the left ventricle increases during isovolumetric contraction, the pressure eventually opens the aortic valve and blood flows from the left ventricle into the aorta, as indicated by reference number 12B. The time at which the aortic valve opens and blood begins to flow from the left ventricle, reference number 12B, is designated in FIG. 1 as T₁ along the time axis 18. FIG. 1 does not illustrate the pressure waveform in the central aorta, but if such waveform were available, the time T₁ would correspond to the foot of the central aortic pulse waveform, and the time interval T₁-T₀ would directly represent the pre-ejection time (PET) interval. However, FIG. 1 illustrates a peripheral pulse waveform 16 (which is measured non-invasively), not a central aortic pressure waveform. The foot 16A of the systolic portion of the peripheral pulse waveform occurs at time T₂, which is delayed and later in time than T₁, see time axis 18. Nevertheless, as long as the patient's peripheral pulse waveform is measured at a fixed location, such as a fixed location on the patient's wrist, the time interval T₂-T₁ is a constant value (K).

It has been found that detecting T₀ corresponding to the first detected peak (14B is the negative peak and 14C is the positive peak) of the LV electrogram is a reliable, accurate method to detected the onset of local ventricular activity. For any given patient sitting during a cardiac resynchronization session, the timing of the negative peak with respect to the positive peak will normally not change. Therefore, detecting the first peak and defining the time interval T₂-T₀ as a surrogate pre-ejection time (SPET) has been found to be accurate and reliable. The detection of the LV electrogram peaks can be accomplished in a number of ways; including identification of the time (T₀) corresponding to the numerical peak value once a certain threshold has been surpassed.

The invention can determine the time T2 (i.e. the foot 16A of the peripheral pulse wave) in various ways. In one method, the foot 16A of the peripheral pulse waveform is calculated as the point where the tangent line of the peripheral pulse upstroke at the maximum second derivative intersects with the tangent line of the of the peripheral pulse before the upstroke. Alternatively, the time T₂ (i.e. the foot 16A of the peripheral pulse wave) can be disclosed in the manner disclosed in U.S. Pat. No. 5,265,011 to O'Rourke, entitled “Method For Ascertaining The Pressure Pulse And Related Parameters In The Ascending Aorta From The Contour Of The Pressure Pulse In The Peripheral Arteries” issuing on Nov. 23, 1993, which is herein incorporated by reference; namely, by analyzing the first derivative of the peripheral pulse waveform and identifying the first negative to positive zero crossing preceding the maximum value for the first derivative. Other suitable methods can be used as well.

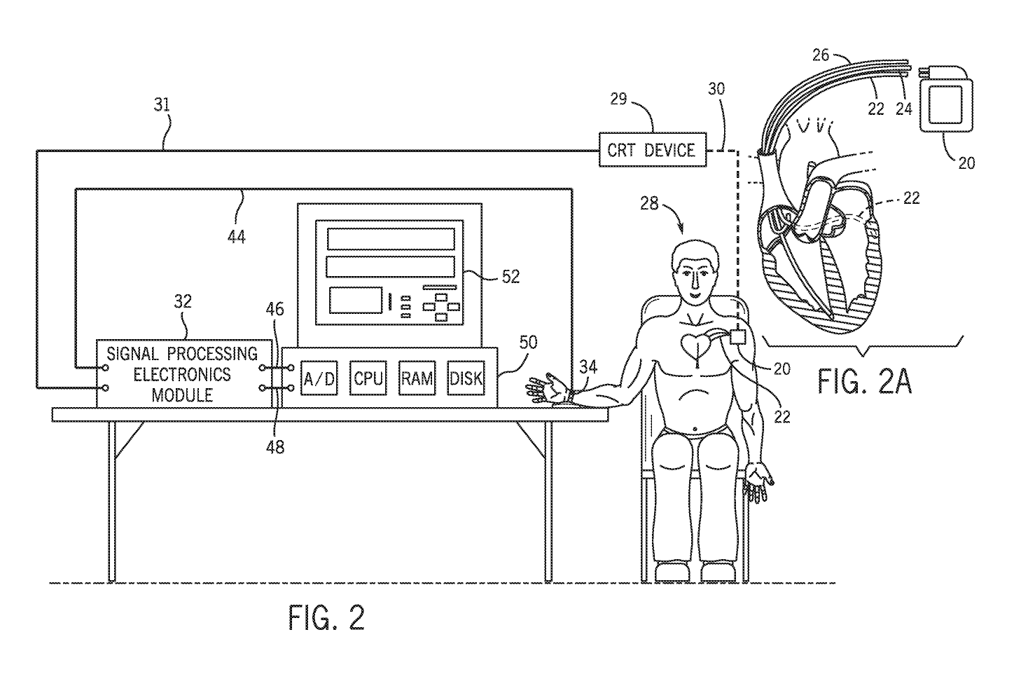

Referring to FIG. 2A, a cardiac pacemaker 20 includes three pacing/sensing leads 22, 24, and 26. The pacing/sensing lead 22 is implanted in the patient's left ventricle. The leads 22, 24, 26 each include an anode and a cathode and are designed to send pacing signals to the heart muscle and sense local electrical depolarization in the heart muscle. The invention can also be implemented with pacemakers having so-called leadless electrodes, See, e.g., U.S. Pat. No. 8,798,745; entitled “Leadless Cardiac Pacemaker System for Usage in Combination with an Implantable Cardioverter-Defibrillator,” by Peter Jacobson. The signal sensed by lead 22 implanted in the left ventricle is transmitted to the cardiac pacemaker 20. The plot of this signal as a function in time is the LV electrogram, see reference number 14 in FIG. 1.

Referring again to FIG. 1, the ejection duration (ED) of the left ventricle is completed when the left ventricle begins to relax and the aortic valve closes. Reference number 12C in FIG. 1 identifies the time (T₃) in which the aortic valve closes on curve 12. Referring now again to the peripheral pulse waveform 16, the waveform includes an incisura 16B, which is a high frequency notch in the waveform 16 resulting from the closure of the aortic valve. Time T₄ on the time axis 18 corresponds to the realization of the incisura 16B in the peripheral pulse waveform 16. While the form of the peripheral pulse waveform 16 is shifted or delayed in time with respect to the central aortic pressure pulse, and also very likely takes on a somewhat different form, see incorporated U.S. Pat. No. 5,265,011, the time interval from the foot 16A of the peripheral pulse wave to the incisura 16B (i.e., T₄-T₂=ED) corresponds accurately to the ejection duration (ED) defining the time interval between the opening 12A of the aortic valve and the closing 12B of the aortic valve. The preferred manner of detecting the location of the incisura 16B in the peripheral pulse wave 16 is disclosed in the above incorporated U.S. Pat. No. 5,265,011; namely, by taking the third derivative of the peripheral pressure waveform and identifying the first positive to negative zero crossing following the largest maximum after a second shoulder in the peripheral pressure waveform unless a second shoulder cannot be identified, in which case the first positive to negative zero crossing following the largest maximum point of the third derivative after the first shoulder.

FIG. 2 is a schematic drawing illustrating the use of the invention. A patient 28 with a pacemaker 20 and LV electrode 22 implanted in the patient's left ventricle has been prepared to undergo CRT optimization. External CRT programming electronics 29 communicate with the pacemaker 20 wirelessly as depicted by dashed line 30, e.g. using a wand or otherwise. The physician will use the CRT device 29 to adjust the settings in the pacemaker 20. The LV electrogram data is transmitted to the CRT device 29. As mentioned, the preferred signal processing electronics module 32 is the SphygmoCor® MM3, manufactured by AtCor Medical. The LV electrogram data is transmitted from the CRT device 29 through cable 31 to the signal processing electronics module 32. FIG. 2 shows an embodiment of the invention in which a tonometer 34 monitors the patient's radial artery pressure waveform. A conventional hand-held tonometer from AtCor Medical is suitable for carrying out the invention, although it is preferred that the tonometer 34 be strapped to the wrist of the patient 24 in order to ensure that the radial pressure pulse wave be taken at a fixed location on the patient's limb during the CRT optimization session. As mentioned, the patient's peripheral pulse waveform can be taken at another location, for example, a brachial artery volumetric displacement waveform can be measured using an upper arm cuff. See, e.g., above incorporated U.S. Pat. No. 9,314,170, entitled “Brachial Cuff”, filed May 5, 2011, by Ahmad Qasem and assigned to the assignee of the present application. The criterion for selecting the type of measured waveform is the ability to identify the foot of the waveform (e.g. 16A in FIG. 1); and, to the extent the invention is used to also measure ED, the ability to identify the incisura of the waveform (e.g. 16B). In any event, it is important that the location of the peripheral pulse waveform be measured at a fixed location during the CRT optimization session in order to ensure that the pressure pulse travel time from the aortic valve to the peripheral measurement location (e.g., wrist, or upper arm) is consistent. Since the invention relies on the simultaneous measurement of an LV electrogram and a peripheral pulse waveform, the accuracy of the invention depends in large part on the measuring of the peripheral pulse waveform at a fixed location.

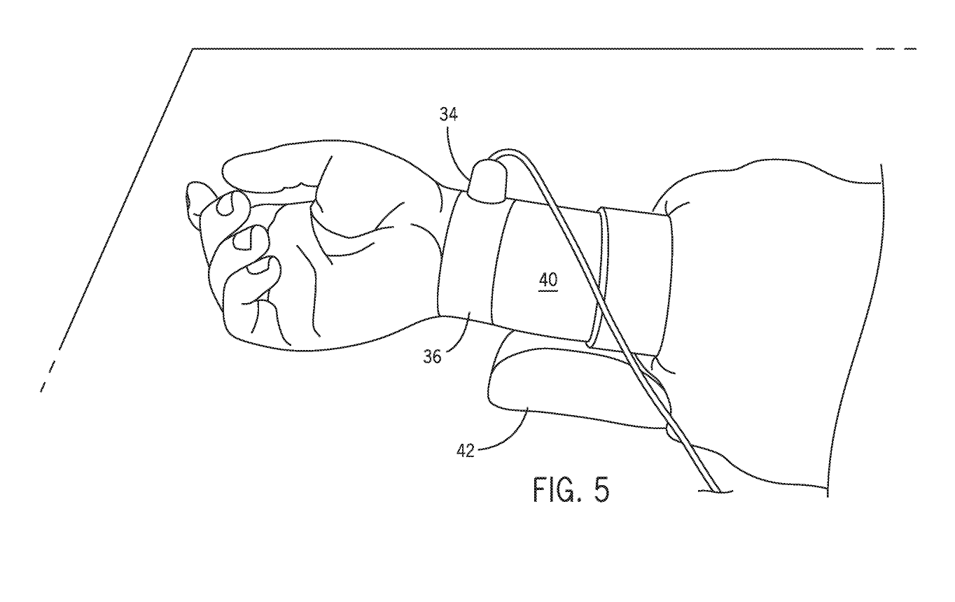

FIG. 5 illustrates a tonometer device that includes a tonometer sensor or transducer 34 attached to a wrist strap 36. As shown in FIG. 5, the strap 36 is preferably wrapped around the patient's wrist 40 so that the face of the tonometer sensor 34 is placed transcutaneously in a fixed location above the radial artery of the patient. Best results are obtained if the wrist is bent outward in the dorsiflex position, which pushes the radial artery towards the surface, thus making it easier to access. When using the dorsiflex position, the wrist 40 should rest on a small cushion 42, as illustrated in FIG. 5. As is known in the art, the pressure of the tonometer against the patient's wrist may have to be adjusted in order to obtain an adequate waveform for the analysis.

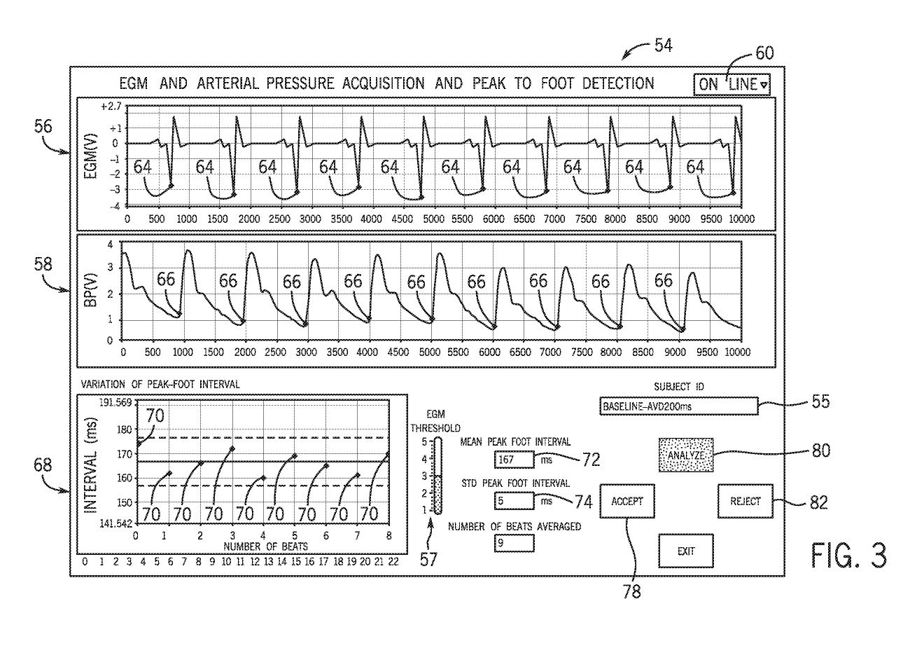

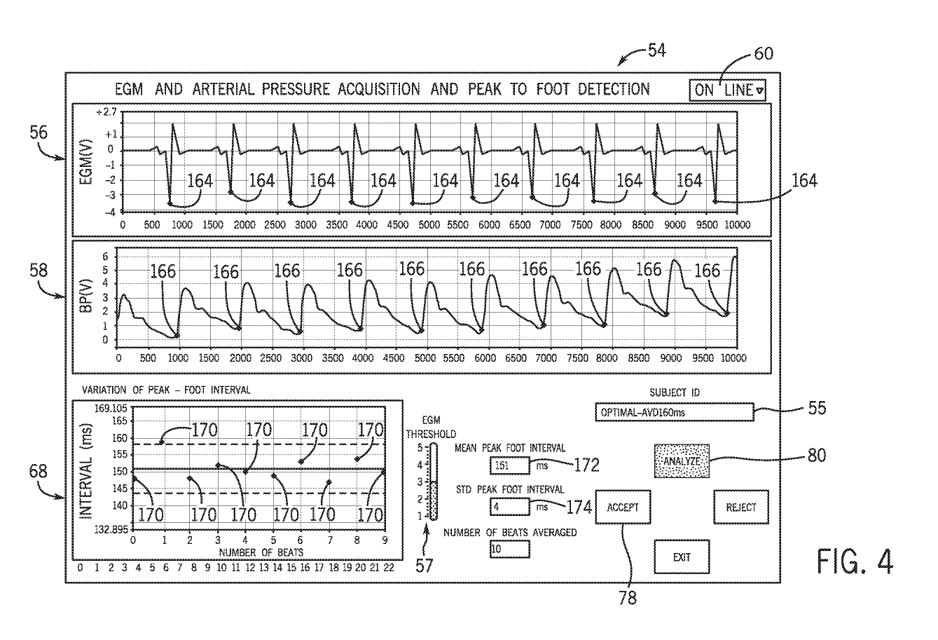

Referring again to FIG. 2, the tonometer 34 (or other suitable pulse waveform sensing device such as a brachial cuff device) is attached to a cable 44 connected to the signal processing electronics module 32. Analog output cables 46 and 48 are connected between the signal processing electronics module 32 and a personal computer 50. LV electrogram data is transmitted in cable 46 whereas peripheral pulse waveform data is transmitted in cable 48. The computer 50 contains an analog to digital converter which receives the analog data in lines 46 and 48 and converts it to digital form. The computer also includes a processing unit, memory, and data storage, as is common in the art. A computer monitor 52 with a screen display is also provided. The computer 50 is programmed with software that displays a trace of the LV electrogram and the peripheral pulse waveform, and also calculates SPET for each waveform pulse, which is referred to in FIGS. 3 and 4 as the “PEAK-FOOT INTERVAL”, as well as trends.

FIGS. 3 and 4 illustrate a computer screen display 54 used to implement the invention. The screen 54 in FIG. 3 contains exemplary data from a patient before CRT optimization. FIG. 4 is similar to FIG. 3; however, the exemplary data is shown for the same patient after CRT optimization. Referring to FIG. 3, patient ID information is entered by the attending staff in box 55 on the screen 54 at the beginning of the session. The screen 54 includes an LV electrogram window 56 (i.e., y-axis labelled EGM(V)) and a peripheral pulse waveform window 58 (i.e., y-axis labelled BP(V)). The screen 54 also includes an offline/online button 60. When the system is online and collecting data, the LV electrogram window 56 displays a trace of the LV electrogram in real-time, and the peripheral pulse waveform window 58 displays a trace of the peripheral pulse waveform, also in real-time. Note that the time scale for the windows 56 and 58 is in milliseconds so the length of the time axis in the windows 56 and 58 corresponds to a ten second interval, which in the case of FIG. 3, corresponds to roughly nine or ten heart beats. The user can set the EGM threshold 57 in order to set the sensitivity for detecting the location of the first positive or negative peak. After startup, the attending physician or staff observes the quality of the EGM trace and peripheral pulse waveform trace while the system is online. When the attending staff believes a quality signal has been obtained, the ANALYZE button 80 is selected to stop data acquisition, and the data in windows 56 and 58 remain stationary. FIG. 3 shows the screen at the time at which the ANALYZE button 80 has been selected. At this point, for each respective heart beat, the system in FIG. 3 determines the peak value of the voltage difference in the LV electrogram 56 and defines the corresponding time as the LV impulse time (T₀) for the contracting ventricle. In EGM window 56, a dot 64 is illuminated on the LV electrogram to indicate the time T₀ corresponding to the peak value of the voltage difference of the LV electrogram for each heart beat.

Also, for each respective pulse, the system in FIG. 3 determines the realization of systolic onset in the detected peripheral pulse waveform and defines the corresponding time as a peripherally measured systolic onset time (T₂). The system places a dot 66 at the foot of the waveform for each pulse.

Screen 54 in FIG. 3 also includes an interval window 68. The interval window 68 displays the variation of SPET (i.e., T₂-T₀=SPET), or the “VARIATION OF PEAK-FOOT INTERVAL”, as labeled in FIG. 3, for each heart beat in the ten second trace captured in windows 56 and 58. The “PEAK-FOOT INTERVAL” (SPET) is illustrated by dots 70 in interval window 68. The system preferably calculates the mean and standard deviation of the calculated SPET values 70 and displays these values on the screen, as indicated by reference numerals 72 and 74. If the attending staff is satisfied with the results of the analysis, the accept button 78 is selected and the data is time-stamped and stored. If the staff is not satisfied that the data is reliable, or for some other reason does not desire to store the information, the reject button 82 is selected.

The system and the information on screen 54 is available for use by the attending physician throughout the process of optimizing the programmable settings for the pacemaker. Button 60 can be selected to take the system offline in order to review past results. FIG. 4 illustrates screen 54, with exemplary data for the same patient as in FIG. 3, but taken after the programmable settings for the pacemaker have been adjusted to optimize cardiac performance. In FIG. 4, reference number 164 depicts the peak value in the LV electrogram captured after optimization. Reference number 166 represents the foot of the respective pulses in the peripheral pulse waveform taken after optimization. In window 68, dots 170 represent the calculated “PEAK-FOOT INTERVAL” (i.e., SPET) for each of the heart beats for the captured ten second period after optimization. Note that reference number 172 in FIG. 4 indicates that the mean “PEAK-FOOT INTERVAL” (i.e., mean SPET) after optimization is 151 milliseconds as compared to 167 milliseconds before optimization, see reference numeral 72 shown in FIG. 3. Also, the standard deviation, reference number 174, is slightly lower after optimization in this example than before optimization. The attending physician or staff can select the accept button 78 to store this data with a time-stamp.

With the invention as described, an attending physician and staff are able to minimize pre-ejection time and presumably isovolumetric contraction time using quantitative data that is collected non-invasively and conveniently. In addition, this data is able to be stored for later use in treating the patient. The accessibility of this data facilitates efficient and faster optimization of pacemaker settings.

As shown in the above incorporated '903 patent, the interval window 68 in FIGS. 3 and 4 can have a user activated toggle to display the patient's calculated ejection duration time (ED) for each pulse. As described above and in the incorporated '903 patent, the time (T₄) corresponding to the realization of the closing of the aortic valve is determined for each respective pulse in the peripheral pulse waveform, and T₄ and T₂ are used to estimate an ejection duration time (ED) for the patient, see FIG. 1. The time T4 corresponds to the incisura (see 16B in FIG. 1), and can be determined, e.g., by taking the third derivative of the peripheral pulse wave and identifying the first positive to negative zero crossing following the largest maximum after a second shoulder in the peripheral pulse wave unless a second shoulder cannot be identified, in which case the first positive to negative zero crossing following the largest maximum point of the third derivative after the first shoulder. The estimated ED values are presented for each pulse, and it may also be desired to display the ration of SPET/ED. The programmer settings for the cardiac pacemaker can then be adjusted in an effort to optimize the value of the ejection duration (ED) for the patient or SPET/ED, as desired.

A data selection window can also be used as shown in the above incorporated '903 patent, which enables the user to select or deselect data to be used in calculating an average surrogate pre-ejection time interval SPET for a given time period.

The foregoing description of the invention is meant to be exemplary. It should be apparent to those skilled in the art that variations and modifications may be made yet implement various aspects or advantages of the invention. It is the object of the following claims to cover all such variations and modifications that come within the true spirit and scope of the invention.

We claim:

1. A method of optimizing one or more programmer settings of a cardiac pacemaker comprising the steps of:
attending to a patient with a cardiac pacemaker having one or more programmable settings wherein at least one pacing/sensing lead of the cardiac pacemaker is implanted in the patient's left ventricle muscle and senses a level of electrical depolarization of the left ventricle;
monitoring the signal sensed by the lead and producing an LV electrogram that plots the amount of electrical depolarization of the left ventricle as a function of time such that local ventricular depolarization is characterized by a deflection in the time plot of the electrical depolarization on the LV electrogram;
simultaneously using a sensor to measure a peripheral pulse waveform of the patient as a function of time;
for each respective pulse, determining from the LV electrogram local depolarization of the contracting left ventricle and defining a time corresponding to the associated deflection as an LV impulse time (Tₒ) for the contracting ventricle;
for each respective pulse, determining the realization of systolic onset in the detected peripheral pulse waveform and defining the corresponding time as a peripherally measured systolic onset time (T₂) for the pulse;
using T₀ and T₂ to calculate a surrogate pre-ejection time interval SPET for the pulse;
presenting information related to the calculated surrogate pre-ejection interval (SPET); and
adjusting one or more of the programmable settings for the cardiac pacemaker in an effort to optimize the value of the calculated surrogate pre-ejection time interval SPET for the patient.

2. A method of optimizing one or more programmer settings of a cardiac pacemaker as recited in claim 1 wherein the step of determining an LV impulse time (Tₒ) comprises determining a first positive peak or a first negative peak in the level of measured depolarization in the LV electrogram for each respective pulse.

3. A method of optimizing one or more programmer settings of a cardiac pacemaker as recited in claim 1 further comprising the steps of:
for each respective pulse, determining the time (T₄) corresponding to the realization of the closing of the aortic valve in the peripheral pulse waveform;
using T₄ and T₂ to calculate an ejection duration time (ED) for the patient;
presenting information relating to the calculated ejection duration time (ED); and
further adjusting one or more of the programmer settings for the cardiac pacemaker in an effort to optimize the value of the ejection duration (ED) for the patient.

4. A method of optimizing one or more programmer settings of a cardiac pacemaker as recited in claim 3 wherein the determined time T₄ for the closing of the aortic valve in the peripheral pulse wave is determined by taking the third derivative of the peripheral pulse wave and identifying the first positive to negative zero crossing following the largest maximum after a second shoulder in the peripheral pulse wave unless a second shoulder cannot be identified, in which case the first positive to negative zero crossing following the largest maximum point of the third derivative after the first shoulder.

5. A method of optimizing one or more programmer settings of a cardiac pacemaker as recited in claim 3 further comprising the step of: calculating and displaying the ratio SPET/ED.

6. A method of optimizing one or more programmer settings of a cardiac pacemaker as recited in claim 1 wherein the settings are adjusted in an effort to minimize the value of the surrogate pre-ejection time interval SPET.

7. A method of optimizing one or more programmer settings of a cardiac pacemaker as recited in claim 1 wherein the peripheral pulse waveform is a radial artery pressure waveform measured by a tonometer.

8. A method of optimizing one or more programmer settings of a cardiac pacemaker as recited in claim 7 wherein the tonometer is strapped to the wrist of the patient in a fixed location to sense the pressure in the patient's radial artery.

9. A method of optimizing one or more programmer settings of a cardiac pacemaker as recited in claim 1 wherein the peripheral pulse waveform is a brachial volumetric waveform measured by a brachial cuff device, where the pressure of a cuff around the patient's upper arm is held constant, and an analog signal from a pressure sensor in the cuff device is filtered to preserve the cardiovascular features of the brachial volumetric pulse waveform.

10. A method of optimizing one or more programmer settings of a cardiac pacemaker as recited in claim 1 wherein a mean value for the surrogate pre-ejection time interval SPET is calculated as the average difference between the determined LV impulse time T₀ and the determined peripheral systolic onset time T₂ for a series of pulses and is presented.

11. A method of optimizing one or more programmer settings of a cardiac pacemaker as recited in claim 1 wherein the onset of systole in the measured peripheral pulse waveform is determined by analyzing the peripheral pulse waveform and identifying a point where a first tangent line of the peripheral pulse upstroke at the maximum second derivative intersects with a second tangent line of the of the peripheral pulse before the upstroke.

12. A method of optimizing one or more programmer settings of a cardiac pacemaker as recited in claim 1 wherein the cardiac pacemaker is a biventricular cardiac pacemaker.

13. A method of optimizing one or more programmer settings of a cardiac pacemaker as recited in claim 1 wherein the LV pacing/sensing lead is a bipolar lead having an anode and a cathode.

14. A method of optimizing one or more programmer settings of a cardiac pacemaker as recited in claim 1 wherein the cardiac pacemaker transmits the signal sensed by the LV pacing/sensing lead to an external programming device.

15. A method of optimizing one or more programmer settings of a cardiac pacemaker as recited in claim 1 wherein the signal is transmitted wirelessly from the implanted cardiac pacemaker to the external programming device.

16. A system to facilitate optimization of programmable cardiac pacemaker settings during cardiac resynchronization therapy, the system comprising:
a sensor adapted to detect a peripheral pulse waveform of a cardiac pacemaker patient;
a screen display; and
a computer processor programmed with software to implement the following steps:
receiving an LV electrogram, said LV electrogram plotting the level of depolarization measured in the left ventricle by an LV pacing/sensing lead as a function of time and where each local ventricular depolarization is characterized by a deflection in the time plot of the LV electrogram;
for each respective pulse on the LV electrogram, determining the time corresponding to deflection in the measured depolarization in the LV electrogram and defining the corresponding time as an LV impulse time (T₀) for a contracting ventricle;
for each respective pulse, determining systolic onset of the detected peripheral pulse wave and defining the corresponding time as a peripherally measured systolic onset time (T₂) for the pulse;
using (T₀) and (T₂) to calculate a surrogate pre-ejection time interval SPET; and
displaying information on the screen relating to the calculated surrogate pre-ejection time interval SPET.

17. A system as recited in claim 16 wherein the sensor is a tonometer.

18. A system as recited in claim 17 wherein the tonometer is mounted to a strap adapted to hold the tonometer against the wrist of a patient in a fixed location to monitor the patient's radial artery.

19. A system as recited in claim 13 wherein the sensor is a brachial cuff device having a pressure sensor to measure the pressure in a cuff wrapped around a patient's upper arm, an output signal, a pump for pumping the cuff to a constant pressure, and suitable high pass and low pass filters that filter the signal from the cuff pressure sensor in order to preserve the cardiovascular features of the brachial volumetric waveform.

20. A system as recited in claim 16 wherein the computer processor is contained within a personal computer onto which the software is loaded; and the system further comprises a digital signal processing electronic module which is electrically connected to the blood pressure sensor and a programming device for the cardiac pacemaker, and provides analog data for the LV electrogram and the peripheral pulse waveform that is transmitted to an analog to digital converter which provides digital data in real-time to the personal computer.

21. A system as recited in claim 16 further comprising a screen display, and further wherein the software displays information on the screen relating to the surrogate pre-ejection time interval SPET.

22. A system as recited in claim 21 wherein the software further analyzes LV electrogram and peripheral pulse waveform data collected over a fixed time period and calculates averages of the SPET for the heart beats within the fixed time period as well as a standard deviation of SPET for the heart beats in the fixed time period.

23. A system as recited in claim 22 wherein the screen display further comprises a data selection window that enables the user to select or deselect data to be used in calculating an average surrogate pre-ejection time interval SPET for a given time period.

24. A system as recited in claim 16 wherein the personal computer is capable of storing patient LV electrogram and peripheral pulse waveform data for later analysis.

25. A system as recited in claim 16 wherein the software provides a graphical representation on the screen display of the patient LV electrogram data and the patient peripheral waveform data, both as a function of time.

26. A system as recited in claim 16 wherein the computer is programmed to determine systolic onset of the detected peripheral pulse waveform by analyzing the peripheral pulse waveform and identifying a point where a first tangent line of the peripheral pulse upstroke at the maximum second derivative intersects with a second tangent line of the of the peripheral pulse before the upstroke.
